(19) United States
(12) Patent Application Publication
(10) Pub. No.: US 2009/0293879 A1
(43) Pub. Date: Dec. 3, 2009

(54) Device and Method for Producing Oxygen

(75) Inventor: Oskar Frånberg, Jarfalla (SE)

Correspondence Address: SCHWEGMAN, LUNDBERG & WOESSNER, P.A., P.O. BOX 2938, MINNEAPOLIS, MN, 55402, US

(73) Assignee: Inhalox Technology AB, Stockholm (SE)

(21) Appl. No.: 12/083,885

(22) PCT Filed: Oct. 17, 2006
(86) PCT No.: PCT/SE2006/050406

§ 371 Date: Aug. 14, 2009

Related U.S. Application Data
(60) Provisional application No. 60/597,740, filed on Dec. 19, 2005.

(30) Foreign Application Priority Data
Oct. 21, 2005 (SE) 0502346-0

(51) Int. Cl. A61M 16/10; B01D 53/047

(52) U.S. Cl. 128/205.27; 96/104; 96/102; 95/11

(57) ABSTRACT

The invention relates to a medical device for producing oxygen, wherein the device comprises means for providing first conditions, and means for changing said first conditions to second conditions, the device being configured to during a charging phase extract oxygen from air by, under said first conditions, bringing said air (A) into contact with an agent (SfF) constituted by a reversibly oxygen-fixating agent, i.e. an oxygen selective material, such that the oxygen of the air is adsorbed by said agent, and to remove nitrogen under said first conditions, and configured to during a discharging phase release the oxygen from the agent by means of changing said first conditions to said second conditions. The invention also relates to a method for producing oxygen for individual medical purposes.

### TECHNICAL FIELD

The present invention relates to a device for producing oxygen according to the preamble of claim 1. The present invention further relates to a method for producing oxygen according to the preamble of claim 55.

### BACKGROUND

In the medical field there is a great need for oxygen for various treatments. Patients suffering from e.g. Chronic Obstructive pulmonary Disease (COPD) need oxygen in their daily lives. There is consequently a need for a portable/mobile system which extracts oxygen from air.

The most common way of providing oxygen today is in the form of gas. This is also the most common mobile system for medical use. A bottle of 2 litres pumped to a pressure of 200 bar gives 400 litre free oxygen and weighs about 4 kg. An average mobile patient has an ordination of approximately 2 litre per minute giving the patient approximately 200 minutes of oxygen from a bottle. The gas is produced in a factory by cooling air A to −190° C. liquefying the oxygen while the nitrogen remains gaseous. The liquid oxygen is vaporized and the produced gaseous oxygen is bottled. In this way the oxygen needs to be delivered to the patients in bottles.

In use, in order to save oxygen an oxygen saver may be used. The oxygen saver only doses gas during inhaling and not continuously which normally is the case. This decreases the dosage up to a tenth of normal gas consumption. This technique only works on conscious patients with a distinct breathing, as it otherwise is not possible to detect the breathing clear enough.

An alternative to gaseous oxygen is liquid oxygen (LOX). Liquid oxygen is oxygen cooled down to −186° C., which provides more gas per volume than gaseous gas, up to 900 litres free gas per litre LOX. The technique is complicated as the system must be kept very cold and consequently expensive.

The most common technique today for stationary home treatment with oxygen is the use of Zeolite, silicon-aluminium crystals, a molecular sieve, having a well defined cavity size and lets the oxygen through more easily than nitrogen as the nitrogen better fits in the cavities. The technique may be described as a filter where the nitrogen gets stuck and the oxygen passes through. This is done in a bed. A previously used bed is regenerated by allowing part of the produced oxygen to reflow through the same, against the current, and bring out the nitrogen previously stuck. This is done in cycles referred to as PSA (Pressure Swing Absorption), VSA (Vacuum Swing Absorption), or TSA (Thermal Swing Absorption). In the PSA cycle fresh air is blown in at high pressure and regeneration is done at ambient pressure. In the VSA the fresh air is blown in at ambient pressure and regeneration is done at vacuum. In the TSA cycle the temperature is raised during regeneration in order for the nitrogen to release easier.

These Zeolite systems today weigh about 25-50 kg in order to produce up to 5 litres of gas per minute. An attempt to provide a portable device is disclosed in EP 1485188. It is a zeolite concentrator with a gas saver weighing 4.5 kg without batteries and being able to produce 0.3 litres of oxygen per minute. This device has a high energy consumption leading to short range mobility.

### OBJECTS OF THE INVENTION

One object of the present invention is to provide a device for producing oxygen, particularly a device suitable for medical purposes, which is portable, i.e. easy to carry by one user, which provides sufficient amount of oxygen per time unit to the user, is light weight, and has a low energy consumption.

Another object of the present invention is to provide a method for producing oxygen which is suitable for medical purposes, particularly portable medical purposes, and which is efficient.

### SUMMARY OF THE INVENTION

These and other objects, apparent from the following description, are achieved by a medical device for producing oxygen and a method for producing oxygen which is of the type stated by way of introduction and which in addition exhibits the features recited in the characterising clause of the appended claims 1, 55. Preferred embodiments of the inventive device and method are defined in appended dependent claims 2-54, 56-58.

By providing a medical device for producing oxygen, wherein the device comprises means for providing first conditions, and means for changing said first conditions to second conditions, the device being configured to during a charging phase extract oxygen from air by, under said first conditions, bringing said air into contact with an agent constituted by a reversibly oxygen-fixating agent, i.e. an oxygen selective material, such that the oxygen of the air is adsorbed by said agent, and to remove nitrogen under said first conditions, and configured to during a discharging phase release the oxygen from the agent by means of changing said first conditions to said second conditions, a more efficient medical device may be designed, which is of lighter weight, is portable and mobile, and produces a sufficient amount of oxygen per time unit such that it advantageously may be used by e.g. a COPD-patient, as the oxygen selective material is 100% selective to oxygen as compared to the zeolite process which partly binds oxygen with the nitrogen. The oxygen constitutes approximately 20% of the air, whereas the nitrogen constitutes approximately 80% of the air. Therefore only a fifth of the space where the gas is adsorbed is needed. This thus facilitates providing a more efficient oxygen production and a lighter device.

Preferably the device comprises the features of the dependent claims 2-54, in which further advantageous embodiments are set out.

By providing a method for producing oxygen for individual medical purposes, which comprises the steps of: during a charging phase, extracting oxygen from air by, under first conditions, bringing said air into contact with an agent constituted by a reversibly oxygen-fixating agent/adsorbent, i.e. an oxygen selective material, such that the oxygen of the air is adsorbed by said agent; and removing the nitrogen of the air; and during a discharging phase, releasing the adsorbed oxygen by controlled change of said conditions to second conditions, a more efficient oxygen production is achieved.

Preferably the method comprises the features of the dependent claims 56-58, in which further advantages are set out.

### DESCRIPTION OF THE DRAWINGS

A better understanding of the present invention will be had upon the reference to the following detailed description when read in conjunction with the accompanying drawings, wherein like reference characters refer to like parts throughout the several views, and in which:

FIG. 1a schematically shows a side view of a device for producing oxygen according to a first aspect of a first embodiment of the present invention;

FIG. 1b schematically shows a side view of a device for producing oxygen according to a second aspect of the first embodiment of the present invention;

FIG. 1c schematically shows a side view of a device for producing oxygen according to a third aspect of the first embodiment of the present invention;

FIG. 1d schematically shows a side view of a device for producing oxygen according to a fourth aspect of the first embodiment of the present invention;

FIG. 2 schematically shows a side view of a device for producing oxygen according to a second embodiment of the present invention;

FIG. 3a schematically shows a view of a device for producing oxygen according to a first aspect of a third embodiment of the present invention;

FIG. 3b schematically shows a view of a device for producing oxygen according to a second aspect of the third embodiment of the present invention;

FIG. 4a schematically shows a side view of a device for producing oxygen according to a fourth embodiment of the present invention;

FIG. 4b schematically shows a rear view of the device for producing oxygen in FIG. 4a;

FIG. 5 schematically shows a side view of a device for producing oxygen according to a fifth embodiment of the present invention;

FIG. 6 schematically shows a side view of a device for producing oxygen according to a sixth embodiment of the present invention;

FIGS. 7a-7d schematically shows a side view of different states of a device for producing oxygen according to a seventh embodiment of the present invention;

FIG. 8a schematically shows a side view of a device for producing oxygen according to an eighth embodiment of the present invention in a charging phase; and

FIG. 8b schematically shows a side view of the device in FIG. 8a in a discharging phase.

### DETAILED DESCRIPTION OF THE INVENTION

The invention discloses different embodiments of a medical device for producing oxygen O₂, wherein the device is configured to extract oxygen by under first conditions bringing air A into contact with a reversibly oxygen-fixating agent S/F, i.e. an oxygen selective material, which may be comprised, e.g. by a metal complex such as, e.g., cobalt-bis-salicylaldehyde-ethylene-diimine (salcomine), cobalt-bis-3-fluoro-salicylaldehyde-ethylene-diimine (fluomine), cobalt-bis-3-ethoxy-salicylaldehude-ethylene-diimine (ethomine), metal complexes in the form of cobalt porphyrines, cobalt shiffbase, or simple inorganic salts, such that the oxygen of the air is fixated/adsorbed by said agent; removing the nitrogen of the air; and releasing the adsorbed oxygen by controlled change of said conditions to second conditions. Preferably salcomine and/or fluomine and/or ethomine are intended to be used. The reversibly oxygen-fixating agent is hereinafter referred to as the agent S/F.

In the oxygen generation process there are consequently two phases, the charging phase where oxygen O₂ in the air A reacts with the agent S/F and is adsorbed, and the discharging phase where the oxygen is released from the agent. The device is configured to in said charging phase bringing air A into contact with a reversibly oxygen-fixating agent/adsorbent under first conditions, i.e. the agent S/F such that the oxygen in the air A reacts with the agent and is adsorbed, at which state the nitrogen N₂ is arranged to be let out, and in said discharging phase release oxygen O₂ by change of said first conditions. Said first conditions may comprise applying increased pressure or applying decreased temperature, i.e. cooling, or a combination of both, and change of said first conditions may comprise reduction of pressure, applying vacuum/negative pressure, or increase of temperature/heating, or combinations thereof. There are thus three main cycles: a pressure cycle, a vacuum cycle and a temperature cycle, and these may be combined.

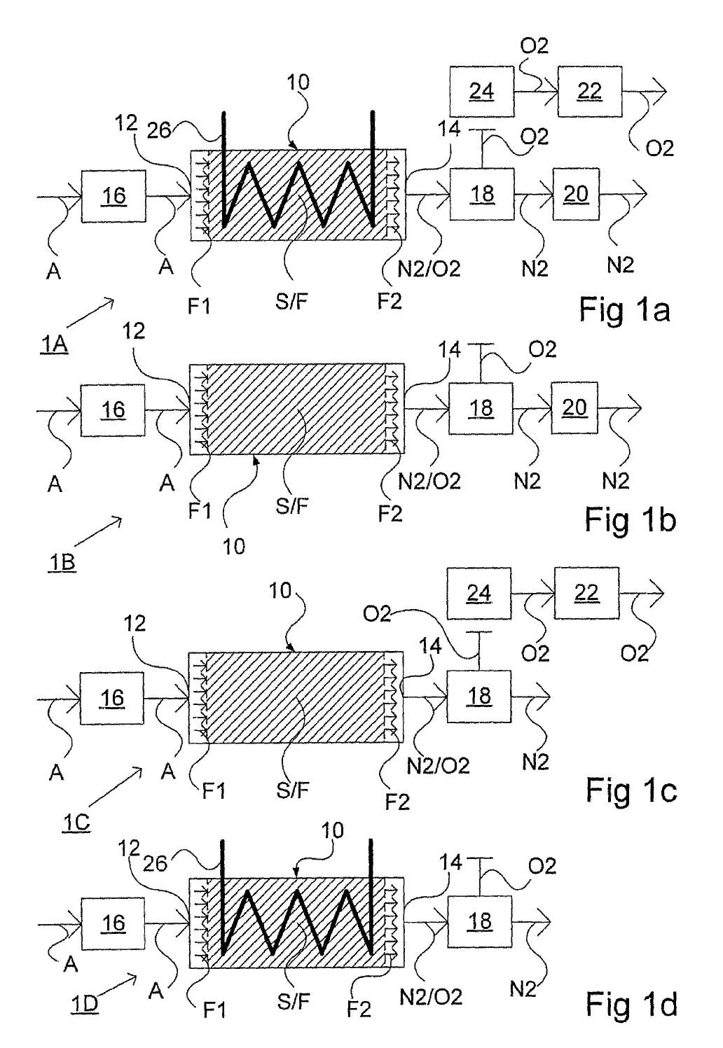

FIGS. 1a-1d show different aspects of a first embodiment of a medical device 1A, 1B, 1C, 1D for generating oxygen O₂ having the common feature of comprising a chamber 10 containing a bed of the agent S/F, the chamber having an inlet 12 for introducing air A into the chamber 10, the agent S/F being arranged in said chamber such that, during the charging phase, the incoming air A reacts with it and oxygen is adsorbed under first conditions, and an outlet for 14, during the charging phase, allowing nitrogen N₂ and possible fractions of oxygen O₂ not adsorbed on the bed of the agent S/F to pass, and during the discharging phase allowing oxygen O₂ released under change of said first conditions to pass. Preferably the device further comprises a first filter means F1 arranged at the air inlet side of the chamber 10 such that air A flowing in through the inlet is filtered, and a second filter means F2 arranged at the outlet side of the chamber 10 such that during the charging phase nitrogen N₂ and possible fractions of oxygen O₂ flowing out through the outlet passes the filter such that possible rests of the agent S/F is filtered, and during the discharging phase oxygen O₂ flowing out through the outlet passes the filter such that possible rests of the agent S/F is filtered. The outlet for nitrogen N₂ and for oxygen O₂ may be the same valve or alternatively two separate valves. Preferably the device comprises isolation means for isolating the chamber 10 such that adiabatic conditions are achieved.

FIG. 1a schematically shows a side view of a device 1A for producing oxygen O₂ according to a first aspect of a first embodiment of the present invention. In this first aspect the device is intended to produce oxygen O₂ by means of a combination of the pressure cycle, the vacuum cycle and the temperature cycle. The device for producing oxygen O₂ comprises the chamber 10, a pressurising means 16, for example fan or compressor arranged to blow air A into said chamber 10 through the inlet 12, a flow selector 18 arranged downstream of the chamber 10, pressure regulation means 20, for example a backpressure regulator arranged to regulate the pressure in the chamber 10 provided downstream of the chamber 10 and preferably downstream of the flow selector 18, depressurising means 22, i.e. means for providing negative pressure in the chamber 10, for example a vacuum pump 22 or the like arranged downstream of the flow selector, and preferably accumulating means 24, for example a vacuum accumulator or depression reservoir arranged upstream of the vacuum pump 22. The device further comprises an outlet for discharging oxygen O₂ to a person/patient. The device further comprises temperature regulation means 26, e.g. a heater/cooler arranged in the chamber 10 and configured to provide cooling during the charging phase and heating during the discharging phase.

During the charging phase air A is arranged to be blown in through the first filter means F1 by means of the compressor or fan. The agent S/F provided in the chamber 10 is arranged to react with the oxygen O₂ of the air A and be bound to the agent S/F. The nitrogen N₂ and possible oxygen O₂ not adsorbed on the bed is arranged to flow out through the outlet 14 of the chamber 10, the second filter means F2 being arranged to filter possible rests of the agent S/F. The nitrogen N₂ and possible fractions of oxygen O₂ is then arranged to be directed by means of the flow selector 18 through the backpressure regulator 20 and discharged at the nitrogen outlet valve 28. The backpressure regulator 20 is at the same time arranged to regulate the pressure in the chamber 10 and keep it at a first pressure level. The temperature regulation means 26 is in this phase arranged to cool the air A in the chamber 10 in order to provide a more effective reaction between the agent S/F and the oxygen O₂. During the discharging phase the oxygen O₂ is arranged to be released by means of controlled change in the conditions. The pressurising means 16, e.g. the compressor 16 or fan 16 is arranged to be shut off such that the pressure in the chamber 10 is reduced. By reducing the pressure the oxygen O₂ may be released, depending on the pressure gradient. A negative pressure is provided in the chamber 10 by means of the vacuum pump 22, the negative pressure providing a more effective release of the oxygen O₂. The released oxygen O₂ is arranged to flow out through the outlet 14 of the chamber 10, the second filter means P2 being arranged to filter possible rests of the agent S/F. The flow of oxygen O₂ is arranged to flow by means of the pressure regulator 20 and by means of the negative pressure created by means of the vacuum, pump 22. The flow of oxygen O₂ is further arranged to be directed by means of the flow selector 18 to the accumulator 24 or alternatively directly to the vacuum pump 22. The chamber 10 is thus arranged to be in flow communication with the vacuum pump 22 via the flow selector 18 and the accumulator 24. The accumulator 24 provides the possibility of accumulating oxygen O₂ if desired. The oxygen O₂ is arranged to be discharged via the oxygen outlet valve arranged at the vacuum pump 22. The temperature regulation means 26 is in this phase arranged to heat the air A in the chamber 10 in order to provide a more effective release of the oxygen O₂.

In the first embodiment the first conditions comprises pressure and cooling, and the changed conditions comprises negative pressure and heating, i.e. a combination of a pressure cycle, a vacuum cycle and a temperature cycle. By applying the different cycles and controlling the conditions thereof the process of producing oxygen O₂ may be optimized, thus achieving an effective oxygen production.

FIG. 1b schematically shows a side view of a device for producing oxygen O₂ according to a second aspect of the first embodiment of the present invention. In this second aspect the device is intended to produce oxygen O₂ by means of the pressure cycle. The device for producing oxygen O₂ comprises the chamber 10, a compressor arranged to blow air A into said chamber 10 through the inlet, a flow selector 18 arranged downstream of the chamber 10, a backpressure regulator 20 arranged to regulate the pressure in the chamber 10 provided downstream of the chamber 10 and preferably downstream of the flow selector 18. The device further comprises an outlet for discharging oxygen O₂ to a person/patient.

During the charging phase air A is arranged to be blown in through the first filter means F1 by means of the compressor. The agent S/F provided in the chamber 10 is arranged to react with the oxygen O₂ of the air A and be bound to the agent S/F. The nitrogen N₂ and possible oxygen O₂ not adsorbed on the bed is arranged to flow out through the outlet 14 of the chamber 10, the second filter means F2 being arranged to filter possible rests of the agent S/F. The nitrogen N₂ and possible fractions of oxygen O₂ is then arranged to be directed by means of the flow selector 18 through the backpressure regulator 20 and discharged at a nitrogen outlet valve. The backpressure regulator 20 is at the same time arranged to regulate the pressure in the chamber 10 and keep it at a first pressure level.

During the discharging phase the compressor is arranged to be shut-off. The oxygen O₂ is arranged to be released by means of controlled reduction of the pressure, which is achieved by means of the pressure regulator 20. The released oxygen O₂ is arranged to flow out through the outlet 14 of the chamber 10 by means of the pressure regulator 20, the second filter means F2 being arranged to filter possible rests of the agent S/F. The flow of oxygen O₂ is further arranged to be directed by means of the flow selector 18 through the pressure regulator 20 and discharged through an oxygen outlet valve.

FIG. 1c schematically shows a side view of a device for producing oxygen O₂ according to a third aspect of the first embodiment of the present invention. In this third aspect the device is intended to produce oxygen O₂ by means of the vacuum cycle. The device for producing oxygen O₂ comprises the chamber 10, means 16 for introducing air into the chamber 10, a flow selector 18 arranged downstream of the chamber 10, depressurising means 22, for example a vacuum pump 22, arranged downstream of the flow selector 18, and preferably a vacuum accumulator 24 or depression reservoir arranged upstream of the vacuum pump 22. The device further comprises an outlet for discharging oxygen O₂ to a person/patient. In addition the device may comprise an additional oxygen accumulator (not shown) intended to be arranged downstream of the depressurising means and means for supplying the oxygen to the user/patient. This would give the possibility to accumulate more oxygen.

During the charging phase air A is arranged to be introduced into the chamber 10 through the first filter means F1 preferably by means of a fan. The agent S/F provided in the chamber 10 is arranged to react with the oxygen O₂ of the air A and be bound to the agent S/F. The nitrogen N₂ and possible oxygen O₂ not adsorbed on the bed is arranged to flow out through the outlet 14 of the chamber 10 by means of the depressurising means 22, e.g. the vacuum pump 22, the second filter means F2 being arranged to filter possible rests of the agent S/F. The nitrogen N₂ and possible fractions of oxygen O₂ is then arranged to be directed by means of the flow selector 18 and discharged at the nitrogen outlet valve provided at the flow selector 18.

During the discharging phase the oxygen O₂ is arranged to be released by applying negative pressure in the chamber 10, by means of the depressurising means 22, e.g. the vacuum pump 22. The released oxygen O₂ is arranged to flow out through the outlet 14 of the chamber 10, the second filter means F2 being arranged to filter possible rests of the agent S/F. The flow of oxygen O₂ is arranged to flow by means of the negative pressure created by means of the vacuum pump 22. The flow of oxygen O₂ is further arranged to be directed by means of the flow selector 18 to the accumulator 24 or alternatively directly to the vacuum pump 22. The chamber 10 is thus arranged to be in flow communication with the vacuum pump 22 via the flow selector 18 and the accumulator 24. The accumulator 24 provides the possibility of accumulating oxygen O₂ if desired. The oxygen O₂ is arranged to be discharged via the oxygen outlet valve arranged at the vacuum pump 22.

FIG. 1d schematically shows a side view of a device for producing oxygen O₂ according to a fourth aspect of the first embodiment of the present invention. In this fourth aspect the device is intended to produce oxygen O₂ by means of the temperature cycle. The device for producing oxygen O₂ comprises the chamber 10, means 16 for introducing air into the chamber, a flow selector 18 arranged downstream of the chamber 10, temperature regulation means 26, e.g. a cooler/heater (temperature regulator 20 comprising cooling means and heating means) arranged to control the temperature in the chamber 10. The device further comprises an outlet for discharging oxygen O₂ to a person/patient.

During the charging phase air A is arranged to be introduced into the chamber 10 through the first filter means F1 preferably by means of the fan. The agent S/F provided in the chamber 10 is arranged to react with the oxygen O₂ of the air A and be bound to the agent S/F. This is achieved by means of temperature regulation means 26, e.g. a heater/cooler which in this phase is arranged to cool the air A in the chamber 10 in order to provide an effective reaction between the agent S/F and the oxygen O₂. The nitrogen N₂ and possible oxygen O₂ not adsorbed on the bed is arranged to flow out through the outlet 14 of the chamber 10, the second filter means F2 being arranged to filter possible rests of the agent S/F. The nitrogen N₂ and possible fractions of oxygen O₂ is then arranged to be directed by means of the flow selector 18 through the same and discharged at the nitrogen outlet valve.

During the discharging phase the oxygen O₂ is arranged to be released by means of controlled increase of the temperature. The temperature regulator 20 (heater/cooler) is in this phase arranged to heat the reacted agent S/F in the chamber 10 in order to provide an effective release of the oxygen O₂. The released oxygen O₂ is arranged to flow out through the outlet 14 of the chamber 10, the second filter means F2 being arranged to filter possible rests of the agent S/F. The flow of oxygen O₂ is arranged to be directed by means of the flow selector 18 through the same and discharged at the oxygen outlet valve.

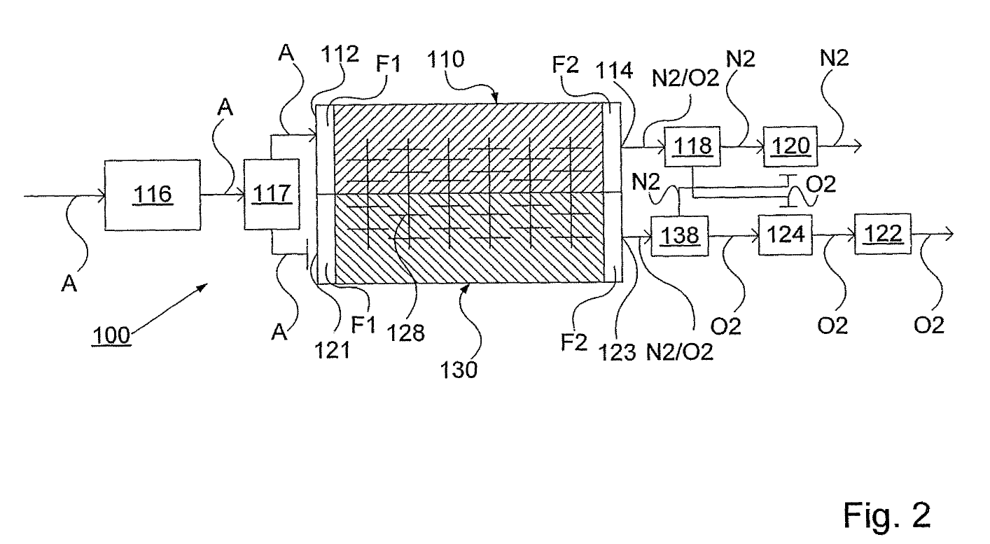

FIG. 2 schematically shows a side view of a device 100 for producing oxygen O₂ according to a second embodiment of the present invention. The device 100 according to the second embodiment provides the same basic function as the device 1A-1D according to FIGS. 1a-1d. A difference compared to the first embodiment is that it comprises at least two chambers 10, 130, each chamber 110, 130 containing a bed of the agent S/F, which facilitates a semi continuous process of oxygen production. In FIG. 2 the device has two beds, but could alternatively have any desired number of beds.

The device comprises a first chamber 110 and a second chamber 130 connected to each other in parallel, each chamber containing a bed of the agent S/F, each chamber having an inlet 112, 121 for introducing air A into the chamber 110, 130, the agent S/F being arranged in said respective chamber 110, 130 such that, during the charging phase, the incoming air A reacts with it and oxygen O₂ is adsorbed under first conditions, and an outlet 114, 123 for, during the charging phase, allowing nitrogen N₂ and possible fractions of oxygen O₂ not adsorbed on the bed of the agent S/F to pass, and during the discharging phase allowing oxygen O₂ released under change of said first conditions to pass. Preferably each chamber comprises a first filter means F1 arranged at the inlet side of the chamber 110, 130 such that air A flowing in through the inlet 112, 121 is filtered, and a second filter means F2 arranged at the outlet side of the chamber such that during the charging phase nitrogen N₂ and possible oxygen O₂ flowing out through the outlet 114, 123 passes the filter such that possible rests of the agent S/F is filtered, and oxygen flowing out during the discharging phase is filtered. In a variant the chambers 110, 130 are isolated by isolation means such that adiabatic conditions are achieved. The device further comprises pressurising means 116, e.g. a compressor 116 or a fan 116, an inflow selector 117 arranged downstream of the pressurising means, arranged to alternately direct the flow to the inlet 112, 121 of the first chamber 110 and the second chamber 130. The device further comprises a first outflow selector 118 arranged to be in flow communication with the first chamber 110, and a second outflow selector 138 arranged to be in flow communication with the second chamber 130. The device 100 also comprises a pressure regulator 120 arranged to be in flow communication with the first and the second outflow selectors 118, 138 alternately, and depressurising means 122, e.g. a vacuum pump 122, arranged to be in flow communication with the first and second outflow selectors 118, 138 alternately, such that when the first outflow selector 118 is in flow communication with the pressure regulator the second flow communicator is in flow communication with the depressurising means 122 and vice versa. Preferably the device further comprises a vacuum accumulator 124 arranged upstream of the depressurising means 122. The device further comprises oxygen O₂ and nitrogen outlet valves. Preferably the chambers are isolated by isolation means. The device preferably comprises heat transfer means configured to be in thermal contact with both beds in the chambers, providing adiabatic conditions during the process.

During the oxygen production process air A is arranged to be blown through the inflow selector 117, said inflow selector 117 being arranged to direct the air A to the first chamber 110, i.e. the charging process is performed in the first chamber 110, where the agent S/F provided in the chamber is arranged to react with the oxygen O₂ of the air A and be bound to the agent S/F. The nitrogen N₂ and possible oxygen O₂ not adsorbed on the bed is arranged to flow out through the outlet of the first chamber 110, the second filter means F2 being arranged to filter possible rests of the agent S/F. The nitrogen N₂ and possible fractions of oxygen O₂ is then arranged to be directed by means of the first outflow selector 118 through the backpressure regulator 120 and discharged at the nitrogen outlet valve. The backpressure regulator 120 is at the same time arranged to regulate the pressure in the first chamber 10 and keep it at a first pressure level. When substantially all oxygen O₂ has been adsorbed on the first bed of agent S/F in the first chamber 110 the discharging process, i.e. the release of oxygen O₂ is intended to start in said first chamber 110. This is done by means of switching the inflow selector 117 arranged such that the air A is directed to the second chamber 130, i.e. the charging process is performed in the second chamber 130.

During the discharging phase in the first chamber 10 the oxygen O₂ is arranged to be released by means of controlled change in the conditions. The first outflow selector 118 is now arranged to be switched to be in flow communication with the accumulator 124 and the depressurising means 122. The oxygen O₂ is arranged to be released by reducing the pressure and providing a negative pressure in the first chamber 110 by means of the vacuum pump 122. The released oxygen O₂ is arranged to flow out through the outlet of the first chamber 110, the second filter means F2 being arranged to filter possible rests of the agent S/F. The flow of oxygen O₂ is arranged to flow by means of the negative pressure created by means of the vacuum pump 122. The flow of oxygen O₂ is further arranged to be directed by means of the first outflow selector 118 to the accumulator 124 or alternatively directly to the vacuum pump 122. The accumulator 124 provides the possibility of accumulating oxygen O₂ if desired. The oxygen O₂ is arranged to be discharged via the oxygen outlet valve arranged at the vacuum pump 122. The discharging process in the first chamber 110 continues until substantially all oxygen O₂ has been extracted.

During the discharging process of the first chamber 110 the charging process takes place in the second chamber 130, the inflow selector 117 is arranged to direct the air A to the first chamber 110, i.e. the charging process is performed in the second chamber 130. This functions in the same way as for the charging process of the first chamber 110, The nitrogen N₂ and possible fractions of oxygen O₂ is here arranged to be directed by means of the second outflow selector 138 through the backpressure regulator 120 and discharged at the nitrogen outlet valve. When substantially all oxygen O₂ has been adsorbed on the second bed of agent S/F in the second chamber 130 the discharging process, i.e. the release of oxygen O₂ is intended to start in said second chamber 130, and at this point of time the inflow selector 117 is arranged to be switched back to direct the flow of air A to the first chamber 110 in which the charging process again is performed. The process thus continues by the switching between the beds. In this example two chambers are connected, but in alternatives more chambers may be connected, such that one or more chambers releases oxygen O₂, one or more beds adsorb oxygen O₂ and one or more chambers are in an intermediate phase.

When e.g. the first chamber is in the charging phase, oxygen reacts with the agent and is adsorbed. The oxygen, as it is adsorbed, emits energy and thus causes heating within the chamber, which negatively affects the adsorption process. The second chamber is then at the same time in the discharging phase, during which the adsorbed oxygen is released. The oxygen, as it is released, receives energy, and thus causes cooling within the chamber, which negatively effects the release process. In order to deal with this problem the device preferably comprises heat transfer means 128 comprising a heat transfer material being arranged in thermal contact with the first and second bed of agent S/F such that when one of the beds adsorbs oxygen O₂ the material is heated due to the reaction energy, and when the other bed releases oxygen O₂ it is cooled due to the reaction energy, and thus an adiabatic process is achieved.

The oxygen production process according to the second embodiment as described above is mainly a pressure process. In such processes a constant temperature is desired in order to avoid temperature differences during charging and discharging. This is achieved according to the adiabatic process above, and thus the process becomes more efficient. Moreover the semi-active process, i.e. the alternating between the chamber makes the process more efficient compared to using a single chamber. As in the first embodiment in FIGS. 1a-1d the different cycles, i.e. the pressure cycle, the vacuum cycle and the temperature cycle, or combinations thereof may be applied.

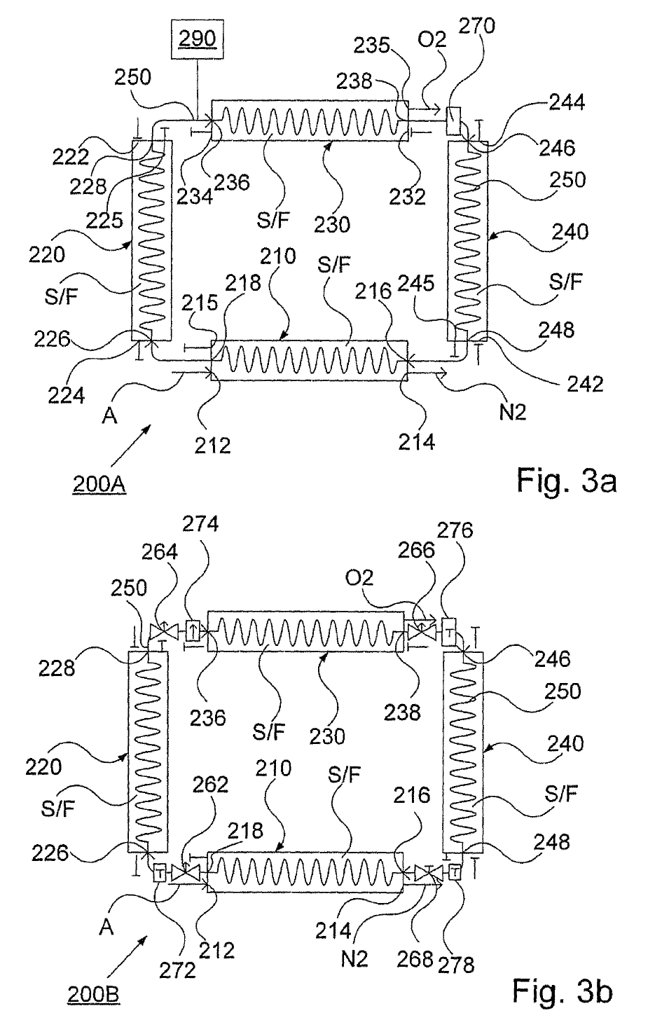

FIG. 3a schematically shows a view of a device 200 for producing oxygen according to a first aspect of a third embodiment of the present invention. This is an alternative to the second embodiment where four chambers are used.

The device 200A comprises a first chamber 210 having an air inlet 212, a nitrogen outlet 214 and an oxygen outlet 215, a second chamber 220 having an air inlet 222, a nitrogen outlet 224 and an oxygen outlet 225, a third chamber 230 having an air inlet 232, a nitrogen outlet 234 and an oxygen outlet 235, a fourth chamber 240 having an air inlet 242, a nitrogen outlet 244 and an oxygen outlet 245, a pipe configuration 250, a heat transfer medium arranged to flow in said pipe configuration 250, and means for transporting the medium, for example a pump 270. Each chamber contains a bed of the agent S/F. Each chamber 210, 220, 230, 240 further comprises an inlet 216, 226, 236, 246 and an outlet 218, 228, 238, 248. The outlet 218 of the first chamber 210 is connected to the inlet 226 of the second chamber 220, the outlet 228 of the second chamber 220 is connected to the inlet 236 of the third chamber 230, the outlet 238 of the third chamber 230 is connected to the inlet 246 of the fourth chamber 240 and the outlet 248 of the fourth chamber 240 is connected to the inlet 216 of the first chamber 210. The chambers 210, 220, 230, 240 are connected by means of the pipe configuration 250, said pipe configuration 250 being arranged through each chamber and creating a continuous flow path through and between the same. The valve means 260 is provided at the pipe 250 between the inlet 216 of the first chamber 210 and the outlet 228 of the second chamber 220. The compressor is provided at the pipe 250 between the outlet 228 of the second chamber 220 and the inlet 236 of the third chamber 230. The device further comprises temperature regulation means arranged to regulate the temperature of the medium in the pipe configuration 250.

The temperature regulation means 290 is arranged to regulate the pressure of the medium in the pipe 250 such that e.g. in the phase shown in FIG. 3a the temperature of the portion of the medium flowing in the first chamber is a low temperature of e.g. 20° C., and the temperature of the portion of the medium flowing at the same time in the third chamber is a high temperature of e.g. 100° C. The portion of the medium at the same time flowing in the second chamber increases from the low temperature to the high temperature, and the portion of the medium flowing in the fourth chamber is cooled down from a high temperature to a low temperature. The process functions such that the charging phase, i.e. oxygen adsorption, is performed in the first chamber 210 as the medium of low temperature at the same time as cooling is performed in the second chamber 220, the discharging phase, i.e. oxygen release, is performed in the third chamber 230, and heating is performed in the fourth chamber 240. The cold medium in the second chamber cools and the charging process starts in this chamber where the medium receive the reaction energy and is heated. The hot medium in the fourth chamber is cooled down and the chamber is heated due to the energy transfer from the medium to the agent in the chamber and the discharging phase starts. As the charging and discharging phases are completed in the first and second chamber respectively there is thus a shift such that the charging phase is performed in the second chamber 220, cooling in the third chamber 230, discharging phase in the fourth chamber 240, and heating in the first chamber. These shifts then continues accordingly when the charging and discharging phases are completed.

FIG. 3b schematically shows a view of a device 200B for producing oxygen according to a second aspect of the third embodiment of the present invention.

All the features in FIG. 3a exists in FIG. 3b except for the feature 270. In addition the device comprises first, second, third and fourth restrictions 262, 264, 266, 268, the first restriction 262 being arranged at the pipe 250 between the first chamber 210 and the second chamber 220, the second restriction 264 being arranged at the pipe 250 between the second chamber 220 and the third chamber 230, the third restriction 266 being arranged at the pipe 250 between the third chamber 230 and the fourth chamber 240, the fourth restriction 268 being arranged at the pipe 250 between the fourth chamber 240 and the first chamber 210. The device also comprises first, second, third and fourth pressurising means 272, 274, 276, 278 respectively arranged at the pipe 250 next to a respective restriction 262, 264, 266, 268.

The device in FIG. 3b functions in basically the same way as the device in FIG. 3a. The difference is that the medium undergoes phase changes, the constrictions and pressurising means are needed to control these phase changes. In the state shown in FIG. 3b where oxygen is adsorbed in the first chamber and oxygen is released in the third chamber the fourth constriction is active, i.e. a restriction is performed such that a negative pressure is created when the medium is pumped through, and the second pressurising means is active. The medium thus undergoes a phase change from liquid phase to gas phase, where the temperature of the medium remains the same during the change. This gives a very efficient process.

The oxygen production process according to the third embodiment as described above is mainly a temperature process. In such processes it is desired to reuse/regain as much as possible of the reaction energy, which is achieved by means of the devices 200A, 200B in FIGS. 3a and 3b.

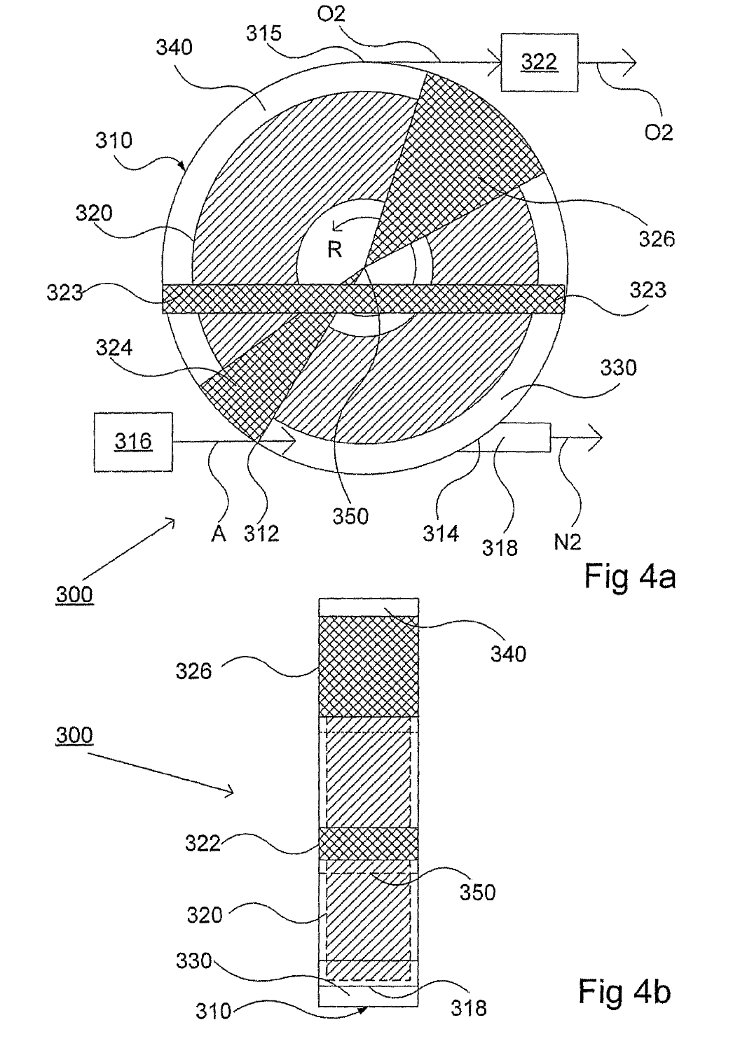

FIG. 4-6 show devices according to different embodiments of a continuous oxygen production process, which devices generally comprises a chamber having a charging region and a discharging region, a rotatable member arranged in said chamber, a drive means arranged to rotate said rotatable member within said chamber, said rotatable member comprising the agent S/F, means for introducing air A into said chamber through an air inlet, means for providing first conditions during a charging phase such that oxygen O₂ of the air A introduced through the inlet reacts and is adsorbed by the agent S/F of the rotatable member, and means for discharging nitrogen N₂ through a nitrogen outlet during said phase, means for providing change of said first conditions such that oxygen O₂ is released from said agent S/F, and means for discharging oxygen O₂ through an oxygen outlet. The oxygen production process is thus continuous, i.e. in the charging region oxygen O₂ of the air A introduced into the chamber is continuously adsorbed by the agent S/F, and in the discharging region oxygen O₂ is continuously extracted and let out for use.

Preferably the device further comprises a first filter means F1 arranged at the inlet side of the chamber such that air A flowing in through the inlet is filtered, a second filter means F2 arranged at the nitrogen outlet of the chamber such that nitrogen N₂ and possible fractions oxygen O₂ flowing out through the outlet passes the filter such that possible rests of the agent S/F is filtered, and a third filter mean arranged at the oxygen outlet of the chamber such that possible rests of the agent is filtered. Preferably the chamber is isolated by isolation means.

FIG. 4a schematically shows a side view of a device for producing oxygen O₂ according to a fourth embodiment of the present invention, and FIG. 4b schematically shows a rear view of the device 300.

The device 300 comprises a chamber 310 having a charging region 330 and a discharging region 340, a rotatable member 320 rotatably arranged in said chamber 310, a drive means 350 arranged to rotate said rotatable member 320 within said chamber 310, the intended rotating direction shown by the arrow R, said rotatable member 320 comprising the agent S/F, pressurising means 316 arranged to blow air A into the charging region 330 of said chamber 310 through an air inlet 312, a nitrogen outlet 314 provided downstream of the air inlet 312 at the charging region, arranged to allow nitrogen N₂ to be discharged, depressurising means 322, for example a vacuum pump 322, provided at the periphery of the chamber 310 substantially opposite to the pressurising means 316 relative to the rotational axis of the rotatable member 320, arranged to suck oxygen O₂ out of the chamber 310 via an oxygen outlet 315, a pressure regulator 318 arranged at the nitrogen outlet, cooling means 324 provided in the charging region 330 upstream of the air inlet arranged to cool the rotatable member 320, heating means 326 provided in the discharging region 340 substantially opposite to the cooling means 324 relative to the rotational axis of the rotatable member 320, and sealing means 323 arranged to maintain a high pressure at the charging region 330 and a low pressure at the discharging region 340.

Preferably the rotatable member 320 has the shape of a circular cylinder, or disk. The chamber 310 is arranged about the rotatable member 320 and preferably has the shape of a hollow circular cylinder. The drive means 350 preferably comprises a drive shaft 312 constituting the axis of the rotatable member 320 and a rotary motor arranged to rotate the rotatable member 320. The cooling means 324 preferably is attached to the chamber 310 at the charging region 330 and the heating means 326 preferably is attached to the chamber 310 at the discharging region 340.

The rotatable member 320 is arranged to continuously rotate in the chamber 310. Cool air A is arranged to be introduced into the charging region 330 of the chamber 310 by means of the pressurising means 316, e.g. a compressor, through the air inlet into the chamber 310 where it is arranged to contact the agent S/F of the rotatable member 320, the air A being arranged to have a pressure and temperature such that it reacts with the agent S/F of the rotatable member 320 and is adsorbed, Nitrogen N₂ and possible fractions of oxygen O₂ is arranged to be discharged through the nitrogen outlet and through the pressure regulator 318. The pressure regulator 318 is arranged to regulate the pressure in the charging region 330 and maintain it at a high pressure. As the rotatable member 320 rotates the portion of the member where oxygen O₂ is adsorbed reaches the discharging region 340 via the sealing means 323. In the discharging region 340, heating means 326 is arranged to heat the rotatble member, and thus the heat the on the agent S/F of the chamber 310. At the same time the depressurising means 322 is arranged to create a negative pressure in the charging region 330. Due to the heating and the negative pressure oxygen O₂ is released from the agent S/F of the rotatable member 320 and is arranged to be let out through the oxygen outlet and through the depressurising means 322, where it is intended to be supplied to a user. As the rotatable member 320 continues to rotate the portion of the member where oxygen O₂ was released reaches the charging region 330 via the sealing means 323. In the charging region 330 the cooling means 324 is arranged to cool the agent S/F of the rotatable member 320 as it passes by. The portion of the rotatable member 320 has now traveled one round along the internal of the chamber 310 and is back at the origin where air A is arranged to be blown in, by means of the pressurising means 316. The cooling of the agent S/F by means of the cooling means 324 and the pressure provided at the agent S/F by means of the pressurising means 316 brings the air A into contact and makes the oxygen O₂ of the air A react with the agent S/F such that oxygen O₂ is adsorbed by the agent S/F.

In the embodiment described above and shown in FIG. 4 a combination of the pressure cycle, vacuum cycle and temperature cycle is used. As in e.g. FIGS. 1a-1d different cycles or combinations of cycles may be used. If a pressure cycle is solely used the heating means 326, the cooling means 322 and the depressurising means 316 are not needed. If a vacuum cycle is solely used the heating means 326, the cooling means 322 and the pressurising means 311 are not needed. If a temperature cycle is used the depressurising means 316 and the pressurising means 311 are not needed.

The oxygen production process according to the fourth embodiment as described above is a continuous process, which increases the efficiency.

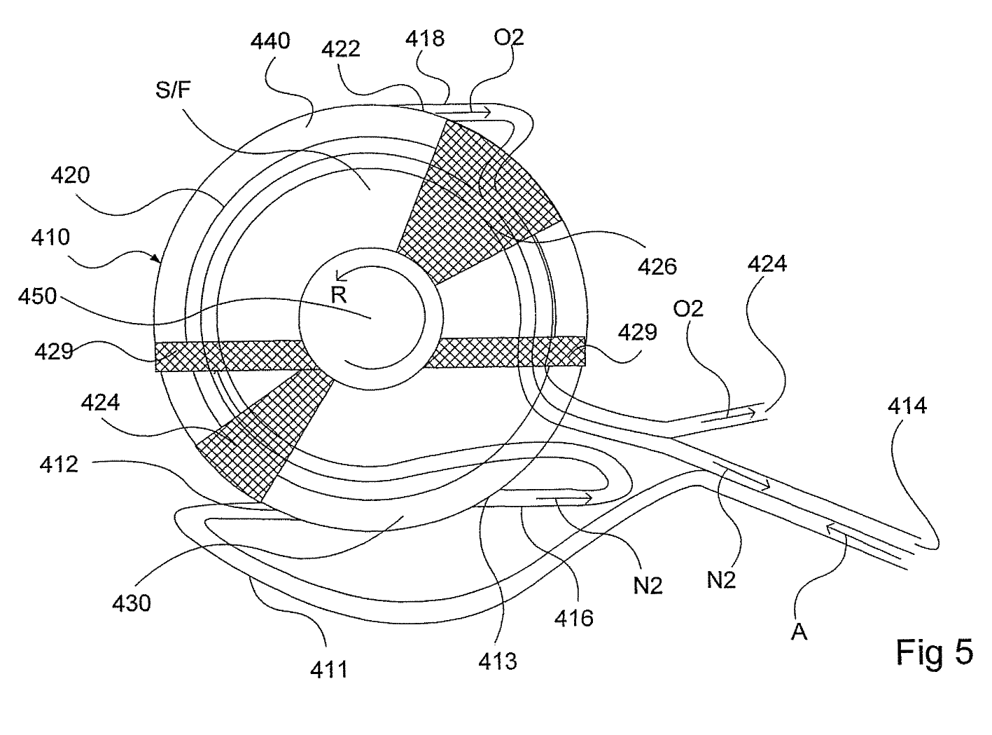

FIG. 5 schematically shows a side view of a device 400 for producing oxygen O₂ according to a fifth embodiment of the present invention. In this embodiment a variant of the temperature cycle of the fourth embodiment is shown.

The device comprises a chamber 410 having a charging region 430 and a discharging region 440, a rotatable member 420 rotatably arranged in said chamber 410, a drive means 450 arranged to rotate said rotatable member 420 within said chamber 410, the intended rotating direction shown by the arrow R, said rotatable member 420 comprising the agent S/F, means for introducing air A into the chamber 410 provided at the periphery of the chamber 410, arranged to blow air A into said chamber 410 through an air inlet 412 of an air heat transfer pipe configuration 411, a nitrogen heat transfer pipe configuration 416 having a nitrogen inlet 413 provided downstream of the air inlet 412 at the charging region 430, and a nitrogen outlet 414 for discharging nitrogen N₂ provided at the end of the pipe, said pipe being arranged along the circumference of the rotable member being in thermal contact with the same, said rotatable member 420 being arranged to rotate relative to the nitrogen heat transfer pipe configuration 416, an oxygen heat transfer pipe configuration 418 having an oxygen inlet 422 provided at the discharging region 440 of the chamber 410 and an oxygen outlet 424 for discharging oxygen O₂ provided at the end of the pipe, said pipe being arranged along a portion of the circumference of the rotatable member 420 adjacent to the nitrogen heat transfer pipe configuration 416. The device further comprises, as in FIG. 4, cooling means 426 provided in the charging region 430 upstream of the air inlet 412 arranged to cool the rotatable member 420, heating means 428 provided in the discharging region 440 substantially opposite to the cooling means relative to the rotational axis of the rotatable member 420, and sealing means 429 arranged to maintain a low temperature at the charging region 430 and a high temperature at the discharging region 440.

Preferably the rotatable member 420 has the shape of a circular cylinder, or disk. The chamber 410 is arranged about the rotatable member 420 and preferably has the shape of a hollow circular cylinder. The drive means 450 preferably comprises a drive shaft constituting the axis of the rotatable member 420 and a rotary motor arranged to rotate the rotatable member 420. The cooling means preferably is attached to the chamber 410 at the charging region 430 and the heating means preferably is attached to the chamber 410 at the discharging region 440.

The rotatable member 420 is arranged to continuously rotate in the chamber 410. Cool air A is arranged to be introduced into an air inlet 412 of an air heat transfer pipe and transported through the same into the charging region 430 of the chamber 410 where it is arranged to contact the agent S/F of the rotatable member 420, the air A being arranged to react with the agent S/F of the rotatable member 420 and be adsorbed. Nitrogen N₂ and possible fractions of oxygen O₂ is arranged to flow into the nitrogen inlet 413 and through the nitrogen heat transfer pipe configuration 416, the flow being against the rotation of the rotatable member 420. As the rotatable member 420 rotates the portion of the member where oxygen O₂ is adsorbed reaches the discharging region 440. The heating means 428 is arranged to heat in the discharging region 440 to a temperature of e.g. 100° C. As the rotatable member 420 continues to rotate the portion of the member where oxygen O₂ was released continues to rotate towards the charging region 430/cooling region. The nitrogen N₂ which is cool in this region and flows against the rotational movement of the rotatable member 420 is arranged to interchange heat with the hot agent S/F of the rotatable member and there will thus be an equalization of temperature in that the hot agent S/F is cooled down and the nitrogen N₂ is heated. The heated nitrogen N₂ is arranged to continue to flow towards the discharging region 440/heating region and is arranged to interchange heat with the cool agent S/F of the rotatable member and there will thus be an equalization of temperature in that the cool agent S/F is heated and the nitrogen N₂ is cooled down. Due to the heating of the agent S/F oxygen O₂ is released from the agent S/F of the rotatable member 420 and is arranged to flow into the oxygen inlet 422 and through the oxygen heat transfer pipe configuration 418 and be discharged through the oxygen outlet 424, where it is intended to be supplied to a user. The oxygen heat transfer pipe configuration 418 in which hot oxygen O₂ is arranged to flow is also arranged such that the agent S/F of the rotatable member 420 is heated. Preferably the oxygen heat transfer pipe configuration 418 is arranged in the discharging region 440/heating region adjacent to the nitrogen heat transfer pipe configuration 416, said pipes being arranged out through the chamber 410 at the charging region 430/heating region along the air heat transfer pipe, providing heat exchange between the oxygen O₂ and nitrogen N₂ and the air A introduced and flowing in the air A pipe, e.g. an optimal temperature for the user, or alternatively to achieve a normal inlet temperature of the introduced air A, or to achieve both. The cooling means 426 is arranged to cool in the charging region 430 to a temperature of e.g. 20° C. The cooling of the agent S/F by means of the cooling means makes the oxygen O₂ of the air A react with the agent S/F such that oxygen O₂ is adsorbed by the agent S/F.

Due to the arrangement of the pipe configurations 411, 416, 418 the oxygen production becomes more efficient compared to a temperature cycle according to the fourth embodiment, as much of the reaction energy is regained.

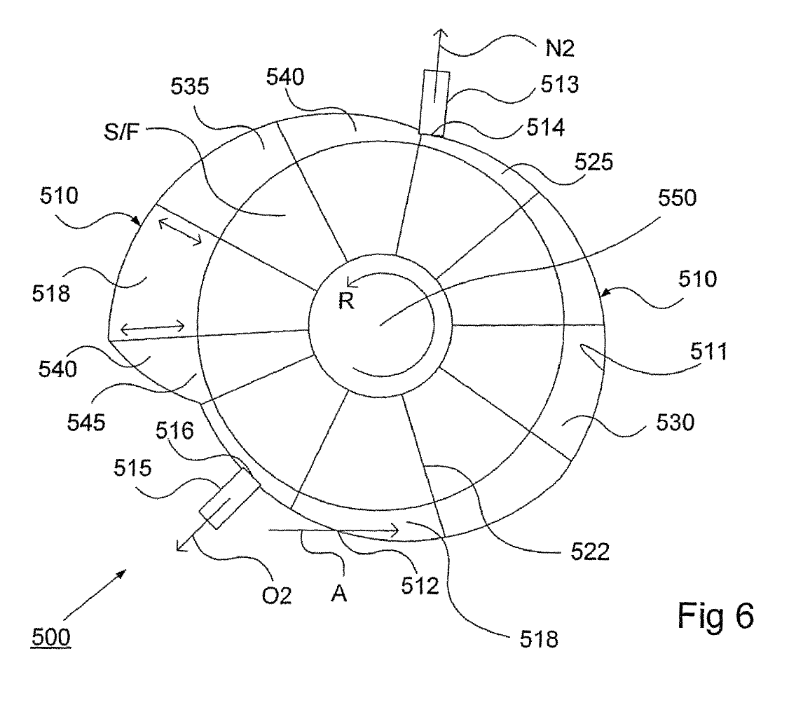

FIG. 6 schematically shows a side view of a device 500 for producing oxygen O₂ according to a sixth embodiment of the present invention. In this embodiment a variant of a pressure and/or vacuum cycle where neither a compressor nor a vacuum pump is needed.

The device 500 comprises a chamber 510 having a charging region 530 and a discharging region 540, a rotatable member 520 rotatably arranged in said chamber 510, a drive means 550 arranged to rotate said rotatable member 520 within said chamber 510, the intended rotating direction shown by the arrow R, said rotatable member 520 comprising the agent S/F, means for introducing air A into the chamber 510 provided at the periphery of the chamber 510. The rotatable member 520 has the shape of a circular cylinder, or disk. The chamber 510 is arranged about the rotatable member 520. The device 500 further comprises flexible sealing means 522, the sealing means 522 comprising a number of sealing partitions 522 or blades protruding substantially radially towards the interior side 511 of the chamber 510 distributed about the chamber 510 such that cavities 518 in the chamber 510 are formed between the partitions 522, each sealing partition being arranged to rotate together with the rotable member. The device 500 further comprises an air inlet 512, a nitrogen outlet 514 arranged at the charging region 530, a first pressure regulator 513 arranged at the nitrogen outlet 514, an oxygen outlet 516 arranged at the discharging region 540, and a second pressure regulator 515 arranged at the oxygen outlet 516. At the air inlet 512 the chamber 510 and two adjacent partitions 522 form a relatively large cavity 518, i.e. the radial distance between the periphery of the rotatable member 520 is relatively large, said distance decreasing slowly along the charging region 530 to the nitrogen outlet 514, thus forming a tapering portion 525 functioning as a constriction, where the discharging region 540 begins and the distance increases relatively more rapidly in a widening portion 535 of the discharging region a certain distance of the discharging region 540 to a region 545 where it decreases even more rapidly substantially to the oxygen outlet 516. The drive means 550 preferably comprises a drive shaft constituting the axis of the rotatable member 520 and a rotary motor arranged to rotate the rotatable member 520.

The rotatable member 520 is arranged to continuously rotate in the chamber 510. Cool air A is arranged to be introduced into a cavity 518 formed by two adjacent partitions 522 of the chamber 510 via the air inlet 512. The introduced air A is thus contained in said cavity 518. As the rotatable member 520 together with the partitions 522 rotate, the cavity 518 moves along the charging region 530, towards the constriction portion 525, the volume of the cavity 518 thus decreasing as the rotatable member 520 rotates, and consequently the pressure in the cavity 518 increases and the oxygen O₂ of the air A reacts with the agent S/F of the portion of the rotatable member 520 within the chamber 510 and is adsorbed. The partitions 522 and the constriction portion 525 constitutes pressurising means as the rotatable member 520 rotates. As the cavity 518 reaches the nitrogen outlet 514 the pressure regulator is arranged to decrease the pressure and nitrogen N₂ is arranged to be discharged through the nitrogen outlet 514. When the cavity 518 reaches the discharging region 540 the volume of the cavity 518 increases as the cavity 518 moves together with the rotatable member 520 along the discharging region 540, and consequently the pressure in the cavity 518 decreases to a negative pressure such that oxygen O₂ is released from the agent S/F of the portion of the rotatable member 520 in the cavity 518. As the rotatable member 520 rotates further the volume of the cavity 518 reaches the pressure normalisation region 545 where the volume of the cavity 518 is arranged to decrease rapidly such that the pressure in the cavity 518 reaches pressure or higher, such that the released oxygen O₂ may be discharged. As the cavity 518 reaches the oxygen outlet 516 the pressure is thus arranged to increase to pressure or higher, and the released oxygen O₂ is then arranged to be discharged through the oxygen outlet 516 through the pressure regulator, to be used by a user. As the rotatable member 520 rotates further, the chamber 510 then once again reaches the air inlet 512 position and the process starts again.

As an alternative variant of the embodiment described above the rotatable member could have a non circular cylindrical shape and the internal of the chamber could have the shape of a hollow circular cylinder which, if suitably dimensioned would achieve basically the same effect as above.

The oxygen production process according to the sixth embodiment as described above is mainly a pressure process. An advantage with this configuration of the device is that there is no need for a compressor or vacuum pump, which reduces the number of parts, which may be cost efficient.

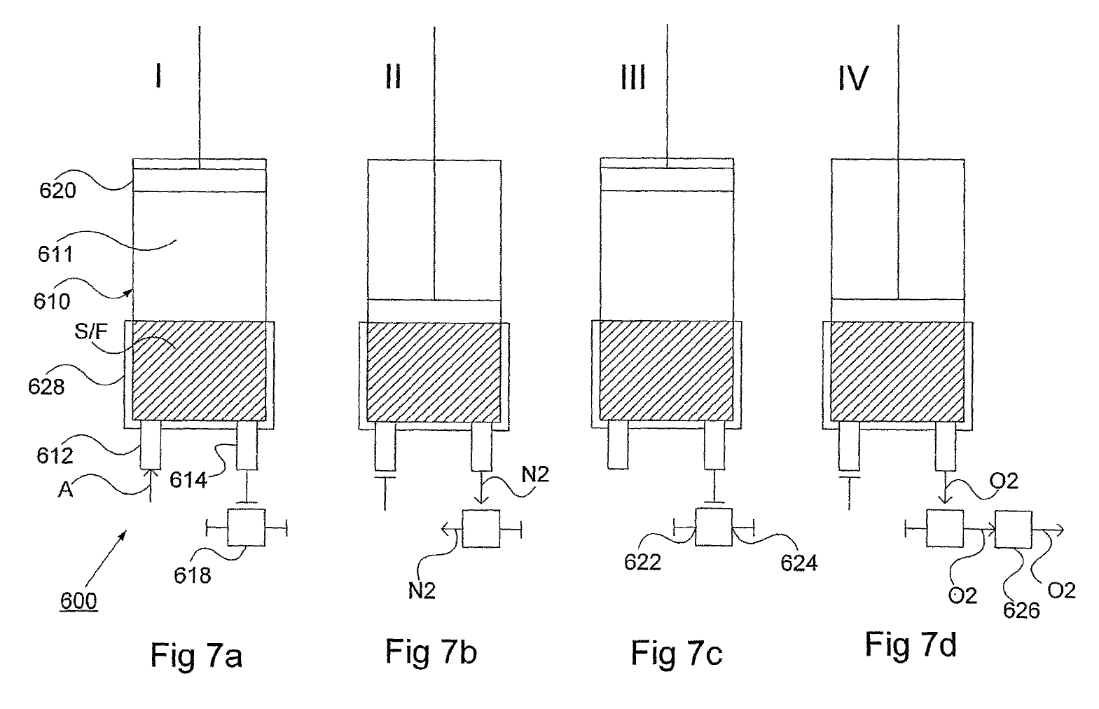

FIGS. 7a-7d schematically shows a side view of different states of a device 600 for producing oxygen O₂ according to a seventh embodiment of the present invention.

The device 600 comprises a chamber 610 having a cylindrical shape, the chamber 610 containing the agent S/F arranged at one end of the chamber 610, a piston 620 provided between the agent S/F and the end of the chamber 610 opposite to the agent S/F end, said piston 620 being reciprocally arranged within the chamber 610 along an axis, between an open position and a closed position, in which open position a cavity 611 is formed between the piston 620 and the agent S/F, and in which closed position the piston 620 is arranged to be at the agent S/F, an air inlet valve 612 arranged at the agent S/F end of the chamber 610 for introducing air A into the chamber 610, an outlet valve 614 arranged at the agent S/F end of the chamber 610 for discharging nitrogen N₂ or oxygen O₂, a flow selector 618 having a nitrogen outlet 622 and an oxygen outlet 624, an accumulator for accumulating oxygen O₂, and a second oxygen outlet 626. The device further comprises isolation means 628 for keeping the device adiabatic.

The oxygen production is performed in four stages I, II, III, IV, where the first and second stage I, II constitute the charging phase, and the third and fourth stage III, IV constitute the discharging phase. Preferably the device 600 comprises isolation means arranged to isolate the chamber 610 or rather the agent S/F of the chamber 610 such that the device 600 may be kept adiabatic during the oxygen production process. Alternatively the device 600 may comprise temperature regulation means 628 arranged to cool at the chamber 610 during the charging phase and arranged to heat at the chamber 610 during the discharging phase.

In the first stage I air A is arranged to be introduced into the chamber 610 through the air inlet valve 612 when the piston 620 is in its open position. At this stage the nitrogen/oxygen outlet valve 614 is closed, as well as the nitrogen outlet 622 and the oxygen outlet 624 of the flow selector 618.

In the second stage II the air inlet valve 612 is arranged to be closed and the piston 620 is arranged to be moved to its closed position, the volume of the cavity 611 decreasing, and the confined air A in the cavity 611 is compressed, consequently increasing the pressure. Due to the high pressure of the air A the oxygen O₂ of the air A reacts with the agent S/F and is adsorbed by the agent S/F. The outlet valve 614 is then arranged to be opened and the nitrogen N₂ of the air A is arranged to flow to the flow selector 618, said flow selector 618 being arranged to direct the nitrogen N₂ to the nitrogen outlet 622 where it is discharged.

In the third stage III the outlet valve 614 is arranged to be closed and the air inlet valve 612 is remained closed. The piston 620 is arranged to be moved to its open position, the volume of the cavity 611 increasing, and negative pressure is created within the cavity 611. Due to the negative pressure the oxygen O₂ is released from the agent S/F.

In the fourth stage IV the outlet valve 614 is arranged to be closed and the air inlet valve 612 is remained closed. The piston 620 is arranged to be moved to its closed position, the volume of the cavity 611 decreasing, and the confined air A in the cavity 611 is compressed, such that the pressure is increased to an ambient pressure or slightly higher. The outlet valve 614 is then arranged to be opened and the oxygen O₂ is arranged to flow to the flow selector 618, said flow selector 618 being arranged to direct the oxygen O₂ to the oxygen outlet 624 where it preferably is arranged to be introduced into an accumulator for accumulating the oxygen O₂, and then be discharged through an outlet of the accumulator for use by a user when desired.

The oxygen production process according to the second embodiment as described above is mainly a pressure process, and is a variant of the process in FIG. 6. Thus, an advantage with this configuration of the device is that there is no need for a compressor or vacuum pump, which reduces the number of parts, which may be cost efficient. This further provides a device which facilitates high oxygen production per time unit due to the reciprocating movement which may reduce the amount of agent/adsorbent material needed.

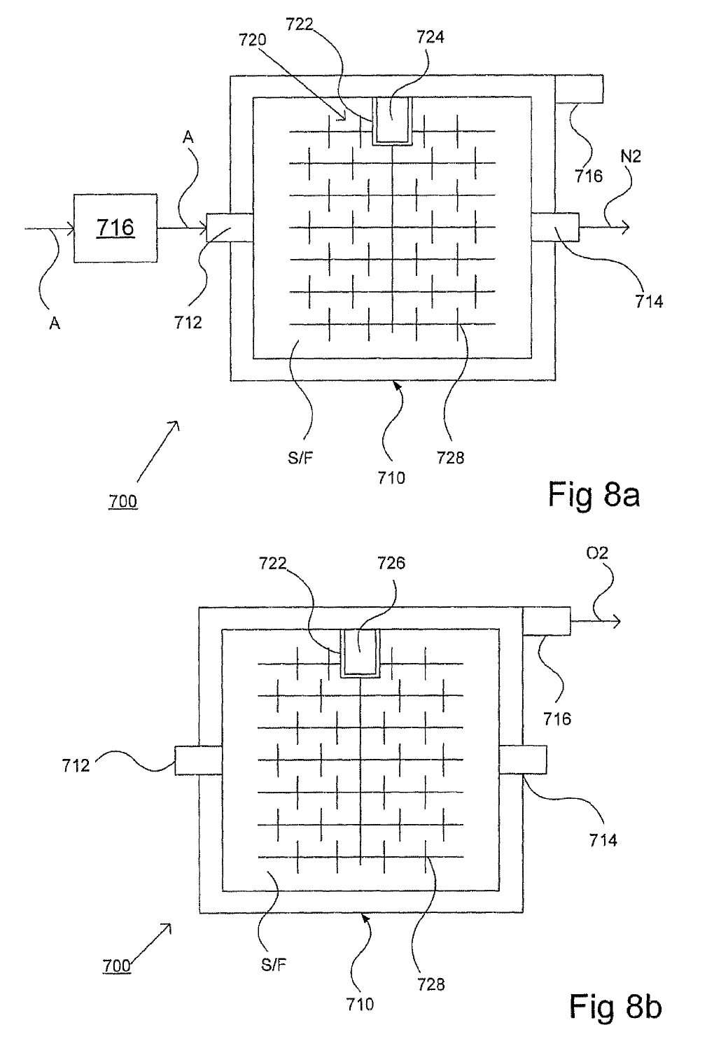

FIGS. 8a and 8b show schematically side views of a device 700 for producing oxygen O₂ according to an eight embodiment of the present invention.

The device 700 comprises a chamber 710, the chamber 710 containing the agent S/F. The device 700 further comprises a connector valve 712 provided at the outside of the chamber 710, a pressurising means 716, for example a compressor or a fan, arranged to be removably connected to the connector valve 712, for supplying air A through said valve into the chamber 710 when connected, a nitrogen outlet 714 arranged at the outside of the chamber 710 for discharging nitrogen N₂, an oxygen outlet 715 arranged at the outside of the chamber 710 for discharging oxygen O₂, temperature regulation means 720 comprising a pocket 722 arranged in the chamber to receive in the charging phase a cooler 724 intended to be removably arranged in said pocket 722, and in the discharging phase a heater 726 intended to be removably arranged in said pocket 722, and heat transferring means 728, for example a flange configuration, being in thermal connection with the cooler 724/heater 726, said temperature regulation means 720 being arranged to cool when the pressurising means 716 is connected to the connector valve 712, and arranged to heat when the pressurising means 716 is disconnected from the connector valve 712. Preferably the device 700 comprises isolation means 726 arranged to isolate the chamber 710.

FIG. 8a shows the device 700 in the charging phase. During the charging phase the pressurising means 716 is arranged to be connected to the connector valve 712. Air A is arranged to be introduced into the chamber 710 by means of the pressurising means 716 via the connector valve 712. The temperature regulation means 720 is arranged to cool the air A and agent S/F in the chamber 710 by means of the cooler 724 being in thermal contact with the heat transferring means 728. The oxygen O₂ of the air A then reacts with the agent S/F and is adsorbed by the agent S/F. Nitrogen N₂ of the air A is arranged to be discharged through the nitrogen outlet 714.

FIG. 8b shows the device 700 in the discharging phase. During the discharging phase the chamber 710 containing the charged agent S/F has been disconnected from the pressurising means 716 by disconnecting the connector valve 712 from the same. The temperature regulation means 720 is arranged to heat the agent S/F, by means of the heater 726 being in thermal contact with the heat transferring means 728. The oxygen O₂ is released from the agent S/F and is arranged to be discharged through the oxygen outlet 715 for use by a user.

Above a device 700 for producing oxygen O₂ using the pressure and thermal, i.e. cooling, function during the charging phase and the thermal, i.e. heating, function during the discharging phase has been described and shown in FIG. 7. However different cycles and functions and combinations thereof may be used.

The device 700 is intended to be used in an ambulance or other applications where oxygen is needed. The rechargeable oxygen storage device is intended to be placed in a charging position in the ambulance when not used, the device then being in a standby position. When needed by e.g. a patient the chamber is removed and oxygen is produced by means of heating such that a patient may be provided with oxygen. When used the chamber is reinstalled in the charging position, charged and ready to be used again. It may thus be reused on site. There is thus no need for having several oxygen bottles which need to be replaced when used or fetch new ones. The chamber may e.g. have the shape of a bottle.

In all embodiments according to the present invention the device may comprises first filter means F1 arranged at the air inlet side of the at least one chamber such that air A flowing in through the inlet is filtered, and second filter means F2 arranged at the nitrogen outlet such that nitrogen N₂ and possible fractions of oxygen O₂ flowing out through the nitrogen outlet passes the filter such that possible rests of the agent S/F are filtered, and arranged at the oxygen outlet such that oxygen O₂ flowing out through the oxygen outlet passes the filter such that possible rests of the agent S/F are filtered.

The device may also in all embodiments comprise an accumulating means 24 arranged to accumulate oxygen discharged through the oxygen outlet. This will enable, for example, giving oxygen continuously even if the production is not continuous, or to be able to give an increased or lowered oxygen flow during a short period if desired by the user.

In the different embodiments different cycles and combinations thereof have been described. Generally, during the charging phase, the charging temperature depends on the pressure such that a high temperature may be used when a high pressure is applied during said phase, i.e. although normally when temperature is mentioned in connection with the charging-phase it is referred to as cooling, but a fairly high temperature may be applied and still get a reaction of the oxygen with the agent given that the pressure is high enough. By cooling is thus meant cooling in a relative term where cooling may be a temperature above room temperature, i.e. above 20° C., for example. Moreover the discharging temperature depends on the negative pressure such that by a negative pressure close to vacuum a low temperature may be used. By heating is thus meant heating in a relative term, where heating may be a temperature below room temperature.

In order to be independent of the ambient temperature the charging temperature may thus be above ambient temperature. An advantage is that temperature shifting in the ambient temperature does not effect the process.

Further the charging pressure may be below normal atmospheric pressure given that the charging temperature is sufficiently low. This may be the case in e.g. an aeroplane or at a high mountain.

The device in all embodiments may also comprise means for controlling the amount of oxygen. The device may for example comprise means for in a controlled way allowing air through the chamber such that air is mixed with the produced oxygen. An advantage is that it facilitates an easy way of controlling the amount of oxygen in e.g. an anaesthesia device. The system can also control the amount of oxygen in a gas mixture by bringing the mixture into contact with the agent S/F. With the agent S/F being at a certain pressure and temperature, the agent would then not only give oxygen to the gas mixture if the oxygen content is too low, but also extract oxygen if the oxygen content is too high. This can be useful, for example, in anaesthesia machines or when diagnosing bow a patient reacts to different oxygen concentrations, or in any condition where it is important not only to have pure oxygen.

The device according to the embodiments of the present invention further facilitates providing pressurised oxygen by means of e.g. pumping an overpressure or increasing the temperature in the bed of the agent.

The device for producing oxygen according to the embodiments above also produces nitrogen. Instead of simply being discharged into ambient air the nitrogen may be used when e.g. an environment which is deficient in oxygen is desired, e.g. by fire/risk of fire by reducing the oxygen content.

The device can also produce gas with an oxygen content lower than that of air. This could be used for any application in which a lower oxygen concentration than normal is useful, for example, for diagnosing patients so see how they react to lower oxygen contents than air, or for athletes breathing air having a lower oxygen content, or people trying to acclimatise to high altitudes by breathing air having a lower oxygen partial pressure than that at sea level.

According to an aspect of the embodiments of the present invention the device may comprise an oxygen saver, which has the advantage that it only doses gas during inhaling and not continuously which normally is the case. This would decrease the dosage up to a tenth of normal gas consumption, and thus facilitate making an even smaller and lighter portable medical device. This technique though has the disadvantage that it only works on conscious patients with a distinct breathing, as it otherwise is not possible to detect the breathing clear enough.

By measuring the level of oxygen uptake and discharge in the agent S/F and using this information to control the uptake and discharge modes the energy consumption in the devices can be lowered and optimized. This also enables the device to handle situations where the ambient air is lower or higher in oxygen content than normal ambient air, for instance if oxygen is to be taken from air where the oxygen content is lower because of some other oxygen consumption nearby or taking oxygen from air at an altitude where the ambient pressure is reduced. Another situation where this may be useful is if the air used is enriched in oxygen, for example, because the unit recovers some of the oxygen exhaled by the patient or if the oxygen content is increased for some other reason.

According to an aspect of the embodiments of the present invention the device may be operated by different energy sources, e.g. battery, mains connection or the like, i.e. a unit which may be operated by battery or other power source, being able to combine these operating processes when a patient is mobile respectively stationary.

According to an aspect of the embodiments of the present invention the device comprises an arrangement which is arranged to control that the agent always is charged to saturation prior to activating the discharge mode, in order to avoid delivering enhanced nitrogen N₂ initially.

According to an aspect of the embodiments of the present invention the device comprises operating modes being adaptable to the needs of the patient, and may for this purpose comprise sensors for detecting breathing based on directly detecting the respiration in or adjacent the respiratory channel, detect changes in the chest, either volume or impedance or ultrasound/light. Or indirect detection by means of measuring the need for oxygen or detecting oxygen saturation in the blood by means of e.g. an optical sensor (e.g. PPG). Alternatively other patient feedback, e.g. connection to nerve signals controlling breathing.

According to an aspect of the embodiments of the present invention the device comprises means, i.e. modes, for signalling when an oxygen dose is required, said means being controllable by the patient.

According to an aspect of the embodiments of the present invention the device comprises means for varying operating modes along the inhalation phase, e.g. oxygen pressure and amount, in order to be received more efficiently by the patient, the modes being individually programmable.

According to an aspect of the embodiments of the present invention the device comprises programming means for programming different breathing modes to be delivered during different states of the patient, e.g. when the patient is awake, asleep, has a change in pulse etc.

According to an aspect of the embodiments of the present invention the device comprises means for delivering a concentrated oxygen dose in case of emergency.

According to an aspect of the embodiments of the present invention the device comprises monitoring means arranged to give information to the patient regarding operation, performance, e.g. of the battery or agent, during operation.

According to an aspect of the embodiments of the present invention the device comprises alarm means with alarm functions having identity depending on type of alarm, i.e. having different alarms alerting to different situations. The alarm functions may be delivered via sound, light or tactile, e.g. vibration. The device may comprise alarm modes being based on prognosis values.

According to an aspect of the embodiments of the present invention the device comprises means for utilizing oxygen not inhaled by the patient, thereby enhancing the life of the chemistry package, i.e. the agent, and reducing the energy consumption.

According to an aspect of the embodiments of the present invention the device may be arranged in direct contact with the body of the patient in order to utilize the body temperature of the patient as a heating source to increase the oxygen release from the agent.

According to an aspect of the embodiments of the present invention the device comprises exchangeable chemistry packages comprising the agent. The chemistry packages, i.e. the agent may be pre conditioned or not pre conditioned.

According to an aspect of the embodiments of the present invention the device comprises a connection arranged to be connected to a patient, said connection being arranged to provide turbulent flow at the oxygen outlet to the patient in order to render the oxygen intake more effective for the patient, i.e. improving the breathing.

1. A device for producing oxygen, comprising means for providing first conditions, and means for changing said first conditions to second conditions, the device being configured to during a charging phase extract oxygen from air by, under said first conditions, bringing said air into contact with an agent constituted by a reversibly oxygen-fixating agent, such that the oxygen of the air is adsorbed by said agent, and to remove nitrogen under said first conditions, and configured to during a discharging phase release the oxygen from the agent by means of changing said first conditions to said second conditions.

2. The device according to claim 1, wherein the device is a portable unit.

3. The device according to claim 1, wherein said means for providing first conditions comprises one or more of the following: pressurising means, cooling means, or a combination of pressurising means and cooling means; and wherein said means for providing controlled change of said first conditions to the second conditions comprises one or more of the following: depressurising means, heating means, or a combination of depressurising means and heating means; where the means for providing first conditions may comprise normal pressure and normal temperature given that the means for providing the second conditions comprises at least one of depressurising means and heating means, and wherein the means for providing second conditions may comprise normal pressure and temperature given that the means for providing the first conditions comprises at least one of pressurising means and cooling means.

4. The device according to claim 1, further comprising at least one chamber arranged to contain a bed of the agent, each chamber having an inlet for introducing air into the chamber, the agent being arranged in said chamber such that, during the charging phase, the incoming air reacts with it and oxygen is adsorbed under first conditions, and a nitrogen outlet arranged to, during the charging phase, allow nitrogen and possible fractions of oxygen not adsorbed on the bed of the agent to pass, and an oxygen outlet to, during the discharging phase, allow oxygen released under change of said first conditions to pass.

5. The device according to claim 4, further comprises means for introducing air into the chamber.

6. The device according to claim 4, further comprising pressurizing means arranged to pressurize the chamber.

7. The device according to claim 6, wherein the pressurizing is provided upstream of the air inlet.

8. The device according to claim 7, wherein the pressurizing means is movably arranged in the chamber such that the air in the chamber is compressed.

9. The device according to claim 4, further comprising a pressure regulator arranged to regulate the pressure in the chamber.

10. The device according to claim 9, wherein the pressure regulator is arranged downstream of the outlet.

11. The device according to claim 10, wherein pressure regulator is movably arranged in the chamber.

12. The device according to claim 4, further comprising depressurizing means, arranged to create a negative pressure in the chamber.

13. The device according to claim 12, wherein the depressurizing means is arranged downstream of the oxygen outlet.

14. The device according to claim 12, wherein the depressurizing means is movably arranged in the chamber such that the air in the chamber is depressed.

15. The device according to claim 4, further comprises a temperature regulator arranged to regulate the temperature in the chamber.

16. The device according to claim 15, wherein the temperature regulator comprises a cooling unit arranged to during the charging phase cool the content of the chamber and a heater during the discharging phase heat the content of the chamber.

17. The device according to claim 16, wherein the temperature regulator comprises a heat transfer arranged to exchange heat in the chamber.

18. The device according to claim 4, further comprises at least two chambers said chambers being arranged such that when at least one of the chambers is in the charging phase, at least one of the other chambers is in the discharging phase, and means for shifting phase when the charging and discharging phases of the respective chambers are finished, such that the at least one chamber is arranged to release the in the charging phase adsorbed oxygen at the same time as one of the other chamber is arranged to adsorb oxygen, and then continues to alternate.

19. The device according to claim 18, wherein at least one of the remaining chambers is in an intermediate phase.

20. The device according to claim 17, further comprises four chambers, each chamber containing the agent, each chamber having an air inlet, a nitrogen outlet and an oxygen outlet, the chambers being connected in series via a heat transfer pipe configuration arranged to receive a heat transfer medium, said medium being arranged to flow through said pipe, and temperature regulation means arranged to control the temperature of the medium such that at the same time oxygen is adsorbed in the first chamber, the second chamber connected downstream relative to the first chamber is cooled, oxygen is released in the third chamber connected downstream of the second chamber, and the fourth chamber is heated, and then shift such that the oxygen is adsorbed in the cooled second chamber, the third chamber is cooled, oxygen is released in the fourth chamber, and the first chamber is heated and so on.

21. The device according to claim 20, further comprises constrictions arranged respectively at the pipe between respective chambers to provide a constriction for the flow of medium prior to the chamber in which oxygen is adsorbed, and pressurizing means arranged respectively at the pipe between respective chambers to provide the flow of the medium.

22. The device according to claim 18, further comprising an inflow selector provided upstream of the air inlets of the at least two chambers arranged to shift the flow of air between them such that a semi continuous oxygen production process is achieved.

23. The device according to claim 4, wherein the chamber comprises a charging region in which the first condition means are arranged to be provided and a discharging region in which the second condition means are arranged to be provided.

24. The device according to claim 23, wherein air inlet and the nitrogen outlet are provided at the charging region, and the oxygen outlet is provided at the discharging region.

25. The device according to claim 23, further comprises a rotating unit arranged to rotate the agent in the chamber such that the agent is brought into contact with the air in the charging region in which, when operated, oxygen is adsorbed, and that the agent is brought into contact with the air in the discharging region in which oxygen is released, such that oxygen is continuously produced.

26. The device according to claim 25, wherein the rotating unit comprises a rotatable member comprising the agent, the agent constituting at least the peripheral portion of the rotatable member, and a drive unit arranged to rotate the rotatable member.

27. The device according to claim 23, wherein the rotating member has a substantially circular cylindrical shape.

28. The device according claim 23, further comprises a seal arranged to provide sealing within the chamber.

29. The device according to claim 28, wherein the seal is arranged to seal such that the first conditions in the charging region and the second conditions in the discharging region are maintained.

30. The device according to claim 28, wherein the seal is arranged to move along with the rotating unit and arranged to seal against the interior side of the chamber such that air is transported along the space formed between the interior side of the chamber and the agent.

31. The device according to claim 15, wherein the temperature regulator is connected to the chamber.

32. The device according to claim 23, wherein a cooling unit is connected to the chamber and arranged to cool in the charging region.

33. The device according claim 23, wherein a heating unit is connected to the chamber and arranged to heat in the discharging region.

34. The device according to claim 17, wherein the heat transfer comprises a nitrogen heat transfer pipe configuration and an oxygen heat transfer pipe configuration arranged relative to each other such that heat exchange is performed in the chamber between the nitrogen, oxygen and reaction energy from the adsorption of and release of oxygen.

35. The device according to claim 34, wherein the heat transfer includes an air heat transfer pipe configuration arranged relative to the nitrogen heat transfer pipe configuration and the oxygen heat transfer pipe configuration such that heat exchange is performed between the nitrogen, oxygen and the air facilitating control of the temperatures of the nitrogen, oxygen, and air.

36. The device according to claim 23, wherein a pressurizing unit is arranged to pressurize in the charging region.

37. The device according to claim 36, wherein the pressurizing unit comprises a seal and a tapering portion of the charging region configured such that when the seal enters the tapering portion the transported air is compressed.

38. The device according to claim 37, the seal comprises at least one of a number of flexible sealing partitions or blades protruding substantially radially towards the internal of the chamber distributed about the chamber such that cavities in the chamber are formed between the partitions or blades.

39. The device according to claim 23, further comprising a discharger to create a negative pressure in the discharging region.

40. The device according to claim 28, wherein the discharging means comprises the seal, and a widening portion of the discharging region configured such that when the seal enters the widening portion the transported air is depressed to a negative pressure.

41. The device according to the pressure regulator comprises a piston arranged in the chamber to provide a reciprocal movement between an open position and a closed position.

42. The device according to claim 41, further comprising a first stage in which the piston is arranged to be in its open position, in which stage air is arranged to be introduced in the chamber, a second stage in which the piston is arranged to compress the air by being moved to the closed position such that oxygen is adsorbed by the agent and nitrogen is arranged to be discharged through the nitrogen outlet, a third stage in which a negative pressure is arranged to be provided by moving the piston to the open position such that oxygen is released, and a fourth stage in which the piston is arranged to be moved to the closed position such that oxygen is discharged through the oxygen outlet.

43. The device according to claim 5, wherein the chamber is removably connected to the air introducing means such that the chamber may be removed from the air introducing means and provide oxygen to a recipient, and be recharged, i.e. arranged to adsorb oxygen, by re-connecting it to the air introducing means after having provided oxygen to a recipient.

44. The device according to claim 5, wherein the air inlet comprises a connector arranged at the external of the chamber, the connector to be connected to the air introducing means during the charging phase, and disconnected from the pressurising means during the discharging phase.

45. The device according to claim 4, further comprises an accumulator to accumulate oxygen discharged through the oxygen outlet.

46. The device to claim 4, further comprises a first filter arranged at the air inlet side of the at least one chamber such that air flowing in through the inlet is filtered, and a second filter arranged at the nitrogen outlet such that nitrogen and possible fractions of oxygen flowing out through the nitrogen outlet passes the filter such that possible rests of the agent are filtered, and arranged at the oxygen outlet such that oxygen flowing out through the oxygen outlet passes the filter such that possible rests of the agent are filtered.

47. The device according to claim 4, further comprises means for providing adiabatic conditions in the chamber.

48. The device according to claim 1, further comprises means for controlling the amount of oxygen.

49. The device according to claim 1, further comprising means for taking care of the nitrogen discharged during the charging phase.

50. The device according to claim 1, wherein said reversibly oxygen-fixating agent is salcomine and/or fluomine and/or ethomine.

51. The device according to claim 1, further comprises an oxygen saver.

52. The device according to claim 1, further comprising a unit for medical purposes.

53. The device according to claim 1, wherein said reversibly oxygen fixating agent is a porphyrine or a cobalt shiffbase.

54. Use of a device according to claim 1 for medical treatment of a patient.

55. Method for producing oxygen, comprising:
during a charging phase, extracting oxygen from air by, under first conditions, bringing said air into contact with an agent constituted by a reversibly oxygen-fixating agent/adsorbent, i.e. an oxygen selective material, such that the oxygen of the air is adsorbed by said agent; and removing the nitrogen of the air; and
during a discharging phase, releasing the adsorbed oxygen by controlled change of said conditions to second conditions.

56. The method according to claim 55, wherein said first conditions comprises any one of the following: high pressure, low temperature, a combination of high pressure and low temperature, or atmospheric pressure and room temperature; and said controlled change of said first conditions to the second conditions comprises anyone of the following: depressurisation, heating, or combinations of depressurisation and heating; where said first conditions may comprise normal pressure, i.e. atmospheric pressure, and normal temperature, i.e. approximately room temperature given that the second conditions comprises depressurising means and/or heating means, and where the second conditions may comprise normal pressure and temperature given that the first conditions comprises pressurising means and or cooling means.

57. The method according to claim 55, wherein said agent is salcomine and/or fluomine and/or ethomine.

58. The method according to claim 55, wherein the discharging provides oxygen for individual medical purposes.
